(19) United States
(12) Patent Application Publication
(10) Pub. No.: US 2024/0189499 A1
(43) Pub. Date: Jun. 13, 2024

(54) DYNAMIC NEGATIVE PRESSURE WOUND THERAPY SYSTEM

(71) Applicant: TAVGER LTD, Katzrin (IL)

(72) Inventors: Michael TAVGER, Katzrin (IL); Irina MARK, Katzrin (IL)

(21) Appl. No.: 18/285,268

(22) PCT Filed: May 13, 2021
(86) PCT No.: PCT/IB2021/054101

§ 371 Date: Oct. 2, 2023

(51) Int. Cl. A61M 1/00

(52) U.S. Cl. CPC A61M 1/92; A61M 2202/0007; A61M 2202/0014; A61M 2202/04; A61M 2202/06; A61M 2205/10; A61M 2205/3331; A61M 2205/3365; A61M 2205/3379; A61M 2205/50

(57) ABSTRACT

Systems and methods for negative pressure wound therapy which provides efficient treatment and a faster healing process. Negative pressure is applied selectively to a part of the wound surface that is most important and/or needs to be treated first. The therapy also enables removing viral components from the wound's surface and preventing cross-contamination through the application of pressure to the wound area by supplying therapeutic fluids in the form of high-pressure micro-jets.

### SCOPE OF THE INVENTION

The presented invention relates to a Negative Pressure Wound Therapy (NPWT) where a Negative Pressure and antiseptic (or other liquid agents) apply to selected parts of the wound through a rigid applicator.

### BACKGROUND OF THE INVENTION

Long-term hard-healing wound care continues to be a challenging problem. Using correct wound care techniques is essential to accelerate healing. The product cost, care time, patient comfort, and infection control issues also are to consider. Extending understanding of wound assessment beyond the wound edge challenges current paradigms of wound healing and has important implications for future approaches to wound assessment. Modern wound care products primarily consist of dressings and skin care products to treat acute and chronic wounds caused by diabetes, immobility, venous disease, traumatic injuries, burns, invasive surgery, etc. When wounds fail to achieve sufficient healing after 4 weeks of standard care, reassessment of underlying pathology and consideration of the need for advanced therapeutic agents should be undertaken. However, the selection of appropriate therapy is often not evidence-based. When choosing a dressing, health care professionals always keep in mind the patient's needs, medical history, environment, and many other factors to consider. The TIME framework summarises the four main components of wound bed preparation: (T=Tissue, non-viable or deficient; I=Infection or inflammation; M=Moisture imbalance; E=Edge of the wound, non-advancing or undermined).

This framework offers practitioners a considered approach to selecting wound interventions by systematically going through each of the components. When used as part of a holistic assessment, it can help practitioners clarify the cause of the problem and facilitate clinical decision-making on how to restore the normal biological environment at the wound bed to promote wound healing.

Apart from wound dressings and medications, other treatments such as a Standard Moist Wound Therapy (SMWT) and Negative Pressure Wound Therapy (NPWT) can also be employed. NPWT provides better efficiency of healing wounds. However, in certain conditions, NPWT is not superior to SMWC (Standard Moist Wound Care), for example, in diabetic foot wounds in clinical practice.

Overall, both have a low wound closure rate. This latter modality is the focus of our invention. NPWT denotes the application of negative pressure to the wound. The purpose of NPWT is to facilitate wound healing, stimulate the wound bed's granulation, and provide the ability to close the wound.

Clinical studies and practices show that providing negative pressure on wound surfaces often speeds up the healing process. Some known methods and devices, [1] are partially utilizing the NPWT method and treating wounds in humans and animals by combining a negative pressure wound therapy (NPWT) with liquid instillation into NPWT device and application of connective cover or/and additional dressing to ensure impermeability over the wound. The main disadvantage of these combined methods is the inability to regulate the magnitude of negative pressure applied to different wound areas during therapy, often necessary for various conditions of different areas of the wound.

There is an invention [2] related to NPWT and offers to instill the element in contact with the wound, providing improved performance and preventing premature insertion of the granule. However, after activating the vacuum system within this method, the porous material is significantly reduced in volume, which generates a tightening force on the wound and undermines the peri-wound skin and deterioration of its healing.

Another device described in [3], operates by using the following combination:
connecting pads for fluid instillation device
negative pressure (NPWT)
wound dressing, as well as a method of attaching an impermeable bandage.

However, in such methods, the fluid given for a wound therapy accumulates in the bandage area where the pressure is the lowest and does not spread deeper into the wound. Insofar as the amount of liquid in the wound is raise, requested more high performance of pump out of the drainage canal.

The next device [4] serves to create and maintain negative pressure at the wound's surface. Within this case, a substantially impermeable air seal is formed around the wound area by a bandage that provides hydraulic communication from the wound area to the source of reduced pressure. The disadvantage of this method is the application of pressure and adhesion of impermeable seal to the skin surrounding a wound area. This creates a problem during the bandage removal and macerates the wound's edges, leading to further complications.

There is an invention [5] related to NPWT and offers to instill the element in contact with the wound, providing improved performance and preventing premature insertion of the granule. However, after activation of the vacuum system within this method the porous material is significantly reduced in volume which generates a tightening force on the wound and undermines the peri-wound skin and deterioration of its healing.

The closest to the invention is a method [6], in which a fluid delivery system (e.g., irrigation fluids and/or medications) can be used in conjunction with Negative Pressure Wound Therapy to accelerate healing and/or recovery. Infected foot wounds are one of the most common reasons for hospitalization and amputation among diabetic patients. The study objective was to investigate a new wound therapy system that employs NPWT with simultaneous irrigation therapy. Unfortunately Data shows [7] that some NPWT devices used in conjunction with irrigation therapy do not provide better results from NPWT used alone.

This method's disadvantage is the poor washing effect of the fluid distribution to the entire surface of the wound due to the lack of turbulent motion of fluid along the surface of the wound, uneven supply of medication to problem areas, especially into the sockets and folds. The applied medical solution is getting mixed with pathogenic secretions from the wound and can transfer an infection from high contaminated areas to less conjugation.

Despite the many and varied existing NPWT systems there are also multiple problems associated with a high risk of bleeding, wound infection, sharp edges in the wound, and serious damage to the periwound skin from adhering to a sealing bandage around the wound.

Negative pressure wound therapy (NPWT) creates a wound environment with below atmospheric pressure on the entire wound surface and stuck skin surface. An impermeable bandage applied to the skin area around the wound is used to maintain the negative pressure. The pump used to maintain negative pressure usually provides pressure between −75 and −125 mm Hg.

The NPWT mechanism is believed to promote wound healing, increase local perfusion, eliminate tissue edema, bring wound edges closer, remove exudate, and inhibit bacterial growth. However, unfortunately, there is no unequivocal evidence and description of the mechanism of vacuum action, which means that there is no way to control the procedure to ensure and obtain the benefits listed above. It can be assumed, for example, that the creation of an area of negative pressure over the wound will cause stretching deformation of the all wound body and then multiple ruptures at the 3D border between relatively healthy tissue beneath and tissue that, because of disease, is losing its integrity above it.

NPWT systems accept a wide range of wound treatment indications. However, there are also known contraindications and risk factors, such as using devices on open organs, exposed vascular network, necrotic tissue with poison, untreated osteomyelitis, malignant neoplasms in the wound, and more.

The NPWT devices have a significant shortage in common—the lack of ability to manage various levels of negative pressure applied to the different wound and periwound skin areas. Beneath the impermeable dressing, the negative pressure acts equally on all fragments of the wound surface, even in those areas where it is contraindicated. The difference of pressures between the wound surface and the distal tissue depth is close to the atmosphere and thus causes deformation and stretching across the body of the wound, leading to rupture inside the wound, bleeding, and slow down the healing process, especially in chronic wounds. Therefore, the maximum vacuum in NPWT is limited to −125 mm Hg. Even in such a vacuum, a wound of 25 cm² experiences a tensile force of 4.3 kg

Stress may also occur in cells in the upper part of the wound due to prolonged contact with the restrictive airtight bandage which can lead to cell death. Further, the devices are unable to determine the exact location and control the intensity of the exudate excretion, which is significantly important during the lengthy procedure. The application of negative pressure to the wound surface increases the difference between the pressure in the capillaries and in the tissues, leading to increased production of exudate, accumulation thereof inside the depressions, maceration of the wound, which cannot be detected under the airtight bandage.

An increased amount of wound exudate can cause damage to the wound bed, decomposition of the intercellular skin matrix around the wound, causing infection. The skin surrounding the wound is an essential resource for wound healing, without which the wounds do not heal. Damage to the skin around the wound can aggravate the pain, increase the wound size and slow the healing process.

The sealing of periwound skin has an additional adverse effect on the skin and its edges. After activating the vacuum, the bandage tightens the skin edges to the center, undermining and splitting the skin from underlying soft tissue. Monitoring the condition of the skin around the wound is an integral part of wound healing.

The periwound skin is the most critical area for the growth of dangerous endogenous anaerobic bacteria and decontamination.

In some embodiments, the NPWT will have an infection control system that feds a detergent or antiseptic solution to the wound's source of infection through the additional tube. However, the only possible driving mechanism for delivering liquids to bacterial plaque in such a scheme considering an impermeable cover is—diffusion. Unfortunately, getting into the field with a pressure gradient, the liquid solution after flowing out from the feeding tube is being immediately mixed with infected exudate from the wound and moved in the opposite direction- to the place where a vacuum is deeper, without actually reaching the sources of infection. The supplied fluid with contaminants absorbed from the wound surface does not come out because of the derivation of a laminar boundary layer on the wound surface, making cleaning even more difficult.

In light of the above limitations, it is desirable to have an improved NWPT mechanism that can facilitate wound healing faster and efficiently without contaminating the surrounding tissue. The NWPT method and device of the present invention described herein comes to address this need.

### SUMMARY OF THE INVENTION

The invention goal is to eliminate the limitations and disadvantages of the known NPWT methods while significantly improving technical characteristics and performance. The negative pressure is applied only to a part of the wound surface, which allows the selective treatment with indications specific to wound areas that are most important and/or need to treat first. For the more efficient treatment and faster healing process, the application of pressure to the wound area is carried out simultaneously with therapeutic fluids supplied in the form of high-pressure micro-jets.

Applying negative pressure to the specific part of the wound and/or periwound skin allows the move of a treatment zone sequentially along the wound's surface using a required speed of motion, change directions, and repeat treatments for each area much that needs.

The invention allows changing the magnitude of the applied negative pressure and pressure of micro-jets with medicinal fluids while simultaneously removing viral components from the wound's surface and preventing cross-contamination.

The invention uses a disposable plastic transparent applicator to apply negative pressure to parts of the wound or skin surface. The contact surface of the applicator is smaller than the wound's size, which is necessary to ensure a high resolution for each wound fragment. It provides an effective treatment for the different conditions and sizes of wound areas—infection, necrosis, excessive exudate, gangrene, etc. After testing, it recommends using a cylindrical applicator with a diameter of a contact sport with a wound of 5 mm.

The applicator of negative pressure installs into the operating distribution handle, which hydraulically connects to vacuum and liquid sources employing elastic tubes with connectors. The operating handle, the applicator, and elastic tubes with appropriative connectors to the liquid supply and drainage outlets all together make a disposable set. During the procedure, the operating handle manually moves so that the applicator is in a local perpendicular position to the wound and makes gentle touches to its surface.

The negative pressure for the treatment and debris aspiration and the high pressure for the fluid supply creates by the peristaltic pumps located in the control console. Flexible occlusion elements of those peristaltic pumps are the same elastic tubes connected to the operating handle. Contaminated liquid after the procedure drains into any empty container or plastic IV bag.

The negative pressure applicator has micro-nozzles designed to inject into its interior high-pressure jets that carry antiseptic liquids or washing solutions without intersection or mixing with hazard slop from the wound surface.

All media—solution, and vacuum—after occlusion in the pumps deliveries to the operation handle via the same flexible tube. Negative pressure occurs inside the applicator attached to the wound. The nozzles for the antiseptic fluid jets are located in the distal bottom of a wound contact zone of the applicator and directly impact the wound and periwound skin area without mixing with contaminated liquid components. The micro-jets size range from 40 to 60 μm is capable to penetrate liquids into hair follicles, sweat gland ducts, and other skin appendages thus preventing the development of endogenous infection in the periwound skin area which is usually colonized by MRSA bacteria.

The operating handle with applicator moves over problem areas of the wound following their profile while providing a negative pressure and supplying the medical liquid for the treatment, disinfection, control excess exudate, wound edges treatment, debridement, removal of foreign particles. The operating handle has 6 degrees of freedom, working as an extension of a human hand, allowing to reach and treat any area of problem shape of the wound surface. In contrast to the known methods NPWT, in this invention, pure antiseptic liquids applied to the area of negative pressure inside the applicator do not mix with the viral media outside of the applicator. Instead, they are situated into the aspiration line immediately after interacting with the wound's surface. The invention allows performing NPWT in full visibility and accessibility to all wounds and surrounding fragments. Changing movement direction while treating the wound and skin surface, allows assessing the condition of the underlying tissues and a degree of homogeneity of parameters at the wounds' edge between the wound and periwound skin. The applicator dimensions allow treating wounds with flat and rugged surfaces which is not always available for existing devices. The invention allows the application of a much deeper negative pressure while moving along the wound surface. Small dimensions of the applicator orifice and its motion features expose the wound surface to strong negative pressure for a short period. Therefore, cell stress does not develop as it is in SMWT and especially in NPWT. Setting stronger negative pressure inside the applicator and making its motion fast allows for expediting the treatment, reducing healing time, and making the procedure more comfortable for patients.

The invention improves vascular function and blood circulation by applying dynamic negative pressure along the main blood vessels located in vulnerable skin areas distant from the wound. The invention stimulates the work of blood vessels and prevents the emergence of new wounds which often occur in various chronic conditions.

The device was designed and manufactured to verify the results and characteristics of wound treatment and compare them to the existing methods based on the proposed method.

The reference will now be made, purely by example, to the accompanying drawings.

The design of the device is shown in the following figures.

### BRIEF DESCRIPTION OF THE DRAWINGS

For a better understanding of the embodiments and to show how it may be carried into effect, reference will now be made, purely by way of example, to the accompanying drawings.

With specific reference now to the drawings in detail, it is stressed that the particulars shown are by way of example and for purposes of illustrative discussion of selected embodiments only, and are presented in the cause of providing what is believed to be the most useful and readily understood description of the principles and conceptual aspects. In this regard, no attempt is made to show structural details in more detail than is necessary for a fundamental understanding; the description is taken with the drawings making apparent to those skilled in the art how the several selected embodiments may be put into practice.

As used in this specification, the singular indefinite articles “a”, “an”, and the definite article “the” should be considered to include or otherwise cover both single and plural referents unless the content clearly dictates otherwise. In other words, these articles apply to one or more referents. As used in this specification, the term “or” is generally employed to include or otherwise cover “and/or” unless the content clearly dictates otherwise.

In the accompanying drawings:

FIG. 1 illustrates a schematic representation of a system including a negative pressure applicator, following an illustrative embodiment of a present invention;

FIG. 2 illustrates a partial sectional view of an operating handle with the negative pressure applicator equipped with micro nozzles for use in the device, following an illustrative embodiment of the present invention;

FIG. 3 is an enlarged schematic representation of the negative pressure applicator equipped with micro nozzles, following an illustrative embodiment of the present invention; and

FIG. 4 presents a series of images of a wound treated with DYNAMIC NPWT.

### DESCRIPTION OF THE SELECTED EMBODIMENTS

Aspects of the present disclosure relate to systems and methods for a DYNAMIC negative pressure wound therapy which provides efficient treatment and a faster healing process. In particular, the therapy involves applying the negative pressure selectively to a part of the wound surface that is most important and/or needs to be treated first. The therapy also enables removing viral components from the wound's surface and preventing cross-contamination through the application of pressure to the wound area by supplying therapeutic fluids in the form of high-pressure micro-jets.

The application of negative pressure to the specific part of the wound and/or periwound skin allows moving of a treatment zone sequentially along the surface of the wound using a required speed of motion, change directions and repeat treatments for each area.

The therapy system of the disclosure allows changing the magnitude of the applied negative pressure and high pressure of micro-jets with medicinal fluids while simultaneously removing viral components from the wound's surface and preventing cross-contamination.

In the therapy system of the present disclosure pure antiseptic liquids when applied to the area of negative pressure inside the applicator do not mix with the viral medium outside of the applicator. Instead, they are removed into the aspiration line immediately after interacting with the wound's surface. The therapy allows an NPWT to be performed in full visibility and accessibility to all wounds and surrounding fragments. It allows to make changes to a required number of passes in selected areas, area's size, and speed enhancing the procedure's efficacy. Further, changing the movement direction when treating the wound surface and the periwound skin allows for an assessment of the condition of the underlying tissues and a degree of homogeneity of their parameters at the edge between the wound and skin. The applicator sizes are chosen with consideration of the characteristic scale of heterogeneity of a wound to allow the processing of a wound and periwound skin with no additional damages. These applicator dimensions allow treating wounds with flat and rugged surfaces which is not always available for existing devices. The therapy also allows the application of a much deeper negative pressure while moving along the wound surface. The small dimensions of the applicator orifice and its motion features expose the wound surface to strong negative pressure for a short period reducing the chances of cell stress. The setting of stronger negative pressure inside the applicator and making its motion faster allows to expedite the treatment, reduce healing time making the procedure more comfortable for patients.

The DYNAMIC NPWT improves vascular function and blood circulation by applying dynamic negative pressure along the main blood vessels located in vulnerable skin areas that are distant from the wound. The therapy system stimulates the work of blood vessels and prevents the emergence of new wounds which often occur in various chronic conditions.

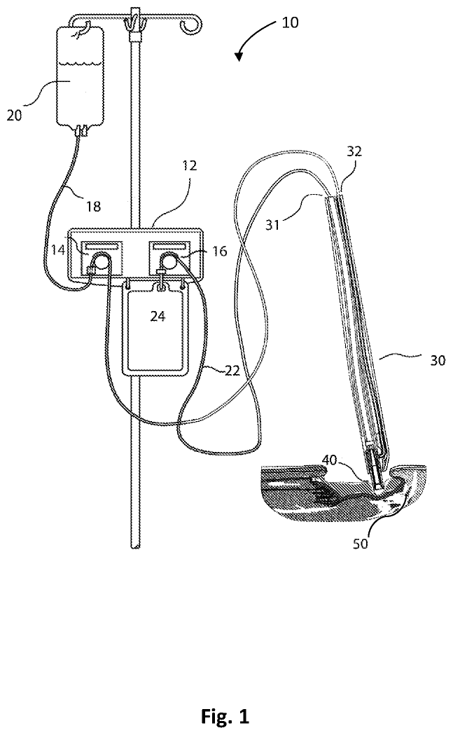

Reference is made to FIG. 1 which illustrates a schematic representation of a system including a negative pressure applicator following an illustrative embodiment of a present invention. FIG. 1 illustrates system 10 which includes a control console 12, two peristaltic pumps including an aspiration pump 16 and an infusion pump 14, an operating handle 30 with a negative pressure applicator 40. System 10 further includes an infusion occlusion tube 18, and a vacuum occlusion tube 22 of the piping system. Both tubes 18, and 22 are introduced into the operating handle 30, located at the distal end of system 10. The operating handle 30 has two ports through each of which the tubes 18, and 22 are connected, respectively. Tube 22 is connected through port 31 of the operating handle 30 while tube 18 is connected through port 32 of the operating handle 30.

The control console 12 is configured to regulate the infusion pump 14 and the aspiration pump 16 to establish the vacuum level in tube 22 connected to port 31. The control console 12 is also configured to regulate the pressure of the liquid in tube 18. The negative pressure applicator 40 is inserted into the operation handle 30 and configured to apply negative pressure on the wound.

During the operation, tube 18 draws the fluid from reservoir 20 and feeds it to the inlet of the infusion pump 14 for supplying pressurized fluid which is further supplied through tube 18 (which is an infusion channel) to port 31. The pressurized fluid is then supplied to the operating handle 30 for wound treatment.

As applicator 40 is introduced to the wound site 50, the pressurized fluid removes the affected particles of the wound material therefrom. Such fluid with wound particles migrates into tube 22 which is connected to outlet port 31 of the operating handle 30. Tube 22 serves as an occlusion element in the peristaltic aspiration pump 16. The aspiration pump 16 communicates with a vessel 24 for storage of hazardous waste after the procedure of NPWT is performed.

The peristaltic pumps 14 and 16 configured to rotate, having variable rotating speed. Such variable rotating speed is configured to control the speed and volume of the aspiration. Since, after exiting the peristaltic pump, the exudate is under atmospheric pressure, it is possible to use a simplified prefabricated tank at atmospheric pressure instead of a pumped-out prefabricated tank. For example, the vessel/collecting bag 24 can be connected to an outlet drain pipe that previously stored a liquid for the treatment.

A vacuum tube can be installed in the vacuum pump and bag 24 for collecting aspirate installed next to the control console 12 before the beginning of the treatment with the negative pressure applicator 40. The infusion peristaltic pump 14 is configured to adjust rotational speeds and control the change of the volume and pressure of the infusion. A simple package or another infusion tank, such as those used for intravenous infusions can be used to deliver the liquid. The infusion tube 18 is provided as a part of a sterile disposable operating handle 30, and a distal end of the infusion tube 18 is mounted on the infusion tank 20, (for example a saline bag). Tube 22 is connected by the end to the operating handle 30 and is connected with a standard connector to the opposite end—the vessel 24 with the contaminated liquid.

The console unit 12 may also be equipped with control switches for switching the infusion pump system 14 and the aspiration pump 16 on and off. Such control functions can be performed in the form of simple on/off switches. Alternatively, systems providing different aspiration and infusion rates can be manually selected by the operator. Such regulation is essential due to differences in the conditions or conditions of the treated wound of specific wound fragments.

The control unit 12, together with the aspiration pump 16, the infusion pump 14, and the corresponding controls, are provided as a separate, reusable unit, for example as a standard control unit. In the illustrated system, control unit 12 is not contaminated because it has no contact with the aspirate during the operation, and the procedure control systems are durable and can be used repeatedly. Control unit 12 can be designed for installation on a portable structure and/or a post for I/O or other structures. According to an aspect of the invention, an NPWT system comprising the operating handle 30, with the negative pressure applicator 40 equipped with micronozzles, tube 22 for aspiration, and tube 18 for infusion can be provided in the form of a sterile single-stranded system.

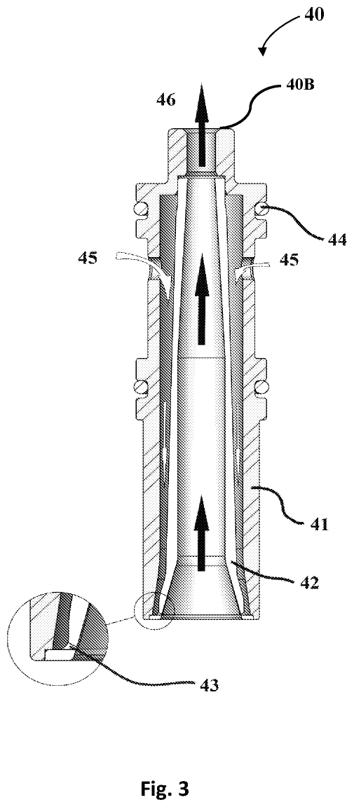

FIG. 3 illustrates a partial sectional view of the operating handle 30 with the negative pressure applicator 40 equipped with micro nozzles 43 for use in the device.

The applicator 40 is installed in the operating handle 30 and can be easily replaced with another one in the event of a blockage of the nozzles. The operating handle 30 is made of two parts, 33 and 34, connecting between themselves after fixing aspiration tube 22 and infusion tube 18. The applicator 40 is installed in the receiving channel of the operating handle 30 and held by the friction forces of the applicator's sealing elements after the start of treatment. The applicator 40 is also held in the operation handpiece due to the difference of negative pressures in the aspiration channel 22.

The aspiration tube 22 is connected by the end to the operating handle 30 and is connected with a standard connector to the opposite end—a vessel 24 with the contaminated liquid.

For vacuum creation in the NPWT system, tube 22 is passed through the peristaltic pump 16 as an occlusion element and then to the handle 30.

A liquid supply tube passes through port 32 and is glued to the side port of part 31 of the receiving channel which is located between the applicator's sealing rings. The medical fluid flows are moving so that their mixing with waste liquid occurs only after high-pressure jets collide with the wound's surface and remove the infected debris and bioburden from its contact area. Mixing a stream of sterile fluid and aspirate inside the operating handle is technically impossible. Solutions supply to the applicator 40 is carried out under pressure. The infusion tube 18 is connected to the operating handle 30 at one end and container 20 with the infusion on the other end and passes through the peristaltic pump 14, as shown in FIG. 1.

FIG. 3 is a schematic representation of a negative pressure applicator 40 equipped with DYNAMIC NPWT micro-nozzles 43. The applicator consists of two parts—the inner 42 and the outer 41 which are glued at the ends forming applicator 40.

The micronozzles 43 may have a diameter of 40-60 u. The micro-nozzles 43 are placed to be at the closest distance from the wound site 50 with the inner side of the applicator 42 in a plane passing through the axis thereof. Under the action of negative pressure, the treated area will be deformed and retracted into a conical part 42 of the applicator 40.

The axis of the micronozzles 43 creates a 90° angle to the surface forming the inner portion of the conical part 41 of the applicator 40 such that the stream emanating from the applicator 40 is perpendicular to the surface of the wound drawn into the applicator 40 under the action of the vacuum.

The infusion tube 18 supplying the liquid, passes through port 32. Tube 18 is glued to the side part 33 of the receiving channel which is located between sealing rings of the applicator 40. The fluid flows are moving in such a way that their mixing occurs only on the surface of the wound. Mixing a stream of sterile fluid and aspirate inside the operating handle is technically impossible. Solutions supply to the applicator 40 can be carried out under pressure.

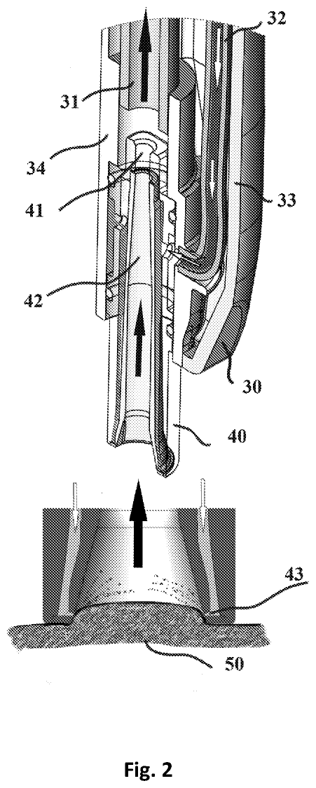

FIG. 3 is a schematic representation of a negative pressure applicator equipped with micro-nozzles. The applicator consists of two parts—the inner 42 and the outer 41. Parts 42 and 41 are glued at the ends forming applicator 40, which has a hollow wall and has 1 or more inlets 45 and 1 or more micro-nozzles with a diameter of more than 40 microns. The micro-nozzles outlets are the closest distance from the wound on the applicator's inner conical surface in a plane passing through its axis. Under negative pressure, the treated area will be deformed and retracted into the conical part of the applicator. It covers the micro nozzles' outlets, as shown in FIG. 2. The axis of the micro-nozzle is directed at a 90° angle to the surface, forming the inner conical part of the applicator. The jets emanating from the applicator's nozzles are perpendicular to the wound surface till the applicator touch the wound surface and the vacuum inside the applicator occurs. After attaching the applicator, the wound tissue will be pressed by vacuum and will block nozzles outlets. The solution pump starts to increase the pressure to overcome force blocking the liquid streaming, and the solution under the pressure will release from nozzles. As the result of the clashing of two forces—vacuum and liquid pressure—space will be created between the wound and applicator, filled with solution. Due to the obstacle of the wound pressed down to the applicator's inner surface and hindering to medical high-pressure liquid injected from nozzles will change the perpendicular direction of its streaming onto parallel inside the gap between the applicator and wound surface obtaining maximum efficacy for cleaning wounds surface from the most difficult for the removal bioburden pluck. To evaluate invention feasibility and compare its efficacy to SMWT and NPWT, the conceptual prototype builts to treat neuro-ischemic foot ulcers with the anamnesis below.

Determinants and estimation of healing
times in neuro-ischemic foot ulcers

| Parameter | SMWT and NPWT | | DYNAMIC NPWT | |
|---|---|---|---|---|
| Initial wound area | 26.6 | mm | 45 | mm |
| Wound area after | 6.25 | mm | 5.8 | mm |
| 10 weeks | | | | |
| Radius of wound | 0.019 | mm/day | 0.034 | mm/day |
| reduction rate | | | | |
| Days for wound | 123.4 | days | 94 | days |
| closing | | | | |
| Number of | 123.4 | days | 10 | |
| treatments | | | | |
| Average treatment | 20 | minutes | 20 | minutes |
| duration | | | | |
| The total duration | 2468 | minutes | 200 | minutes |
| of all treatments | | | | |

| | | |
|---|---|---|
| Treatments | Everyday | Three treatments per month in |
| frequency | dressing change | two first months then two per |
| | | month in the next two month |

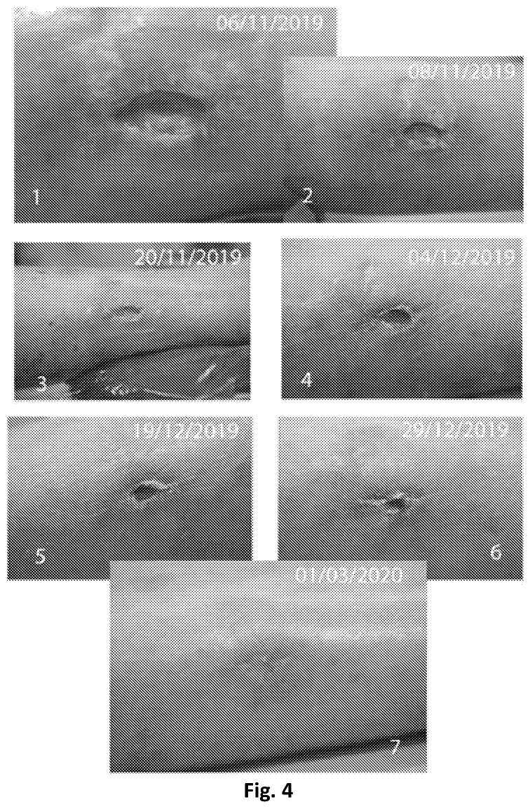

FIG. 4 presents a series of images of a wound treated with DYNAMIC NPWT in 10 treatments over a 94 day period. It is noted that NPWT has significant advantages over all other chronic wound treatments, healing and closing wound 12 times faster and saving 38 hours of operation time by spending only 3 hours for the neuro-ischemic wound treatment.

The DYNAMIC NPWT results recorded in FIG. 4 and the above table achieved in 10 visits compared with 123 visits, which much more beneficial for patients and physicians.

1-13. (canceled)

14. A method for treatment of a wound comprising:
applying negative pressure (TNP) to the wound;
supplying healing fluids to the wound; and
removing foreign particles, contaminated liquids, and wound secretions from a treatment area,
wherein the treatment area surface is smaller than the wound surface.

15. The method of claim 1 further comprising regulating strength of the negative pressure applied to the wound.

16. The method of claim 1 wherein therapeutic fluids are supplied to the wound under increased pressure.

17. The method of claim 3 further comprising regulating strength of the increased pressure at which therapeutic fluids are supplied.

18. The method of claim 3 wherein the steps of supplying healing fluids to the wound and removing foreign particles are performed concurrently.

19. The method of claim 1 further comprising adjusting the treatment area within the surface of the wound.

20. The method of claim 6 further comprising adjusting the treatment area within periwound skin surrounding the wound.

21. A method for wound treatment through a mobile negative pressure wound therapy, the method comprising:
drawing a fluid from a reservoir;
creating a high-pressure fluid for supplying 406 the fluid to a negative pressure applicator, wherein the negative pressure applicator is in fluid communication with a proximal end of an operating handle for healing a wound surface;
applying a controlled amount of the fluid on the wound surface through a negative pressure by the negative pressure applicator;
varying a magnitude and direction of the negative pressure; and
draining the fluid contaminated with wound particles.

22. The method according to claim 21, wherein the process of applying the fluid on the wound and peri-wound skin surface is done using a plurality of micronozzles connected to the negative pressure applicator, wherein the applicator with the plurality of micronozzles has a size smaller than that of a wound surface area under treatment.

23. The method according to claim 22, wherein the micro nozzles are configured to create an angle of 90 degrees with respect to a surface forming an inner conical part of the negative pressure applicator such that the fluid stream emanating from the plurality of micronozzles of the negative pressure applicator is perpendicular to the surface of the wound.

24. The method according to claim 21, wherein the process of drawing the fluid from the reservoir is performed by an infusion pump.

25. The method according to claim 21, wherein varying the magnitude and direction of the negative pressure is done to control the speed and volume of aspiration.

26. The method according to claim 21, wherein applying the controlled amount of the fluid on the wound surface comprises providing a controlled amount of liquid/medication and vacuum to the wound surface under pressure in a vacuum atmosphere.

27. The method according to claim 26, wherein the magnitude of the applied vacuum is significantly lower than 125 mm at an exposure time of a contact area of 1-2 seconds, without developing cellular stress and cell apoptosis.

28. The method according to claim 26, wherein the application of vacuum and pressurized fluid by the negative pressure applicator to the wound surface is carried out by moving the operating handle along the surface by changing the speed and the direction of movement.

29. A system for wound treatment through a mobile negative pressure wound therapy, the system comprising:
a control unit comprising two peristaltic pumps, said control unit configured to regulate operation of the pumps and wherein one of the peristaltic pumps is configured for creating high fluid pressure for supplying a fluid to a wound surface and the other pump is configured to apply negative pressure on the wound;
an elongated operating handle configured to be in fluid communication with the peristaltic pumps, wherein the handle comprising a central aspiration channel and a side infusion channel defining two ports through which a vacuum occlusion tube and an occlusion infusion tube connect said handle with said peristaltic pumps of the control unit;
a disposable transparent applicator configured to be in fluid communication with said two ports, said applicator installed in a receiving channel of the operating handle and held in place by the friction forces of an applicator sealing elements and due to difference of negative pressures in the aspiration channel, wherein the applicator comprises:
a cylindrical hollow body formed by an inner part and an outer part glued at their ends, said inner part defining an axial cylindrical channel with a conical surface at a distal end thereof forming an inner conical part of the applicator hermetically contacting a part of the wound surface; and
a plurality of micronozzles disposed around wide portion of the conical surface which contacts the wound surface and configured to supply pressurized therapeutic fluid micro-jets delivered via said hollow body in fluid communication with the infusion channel to remove contaminations from the wound and peri-wound skin,
wherein the axial channel of the cylindrical hollow body is disposed in direct communication with the central aspiration channel of the handle to apply negative pressure on the wound causing the treated area to be deformed and retracted into said conical part, thereby obtaining the therapeutic level of negative pressure above the wound and to aspirate the contaminated fluid containing therefrom.

30. The system according to claim 29, wherein one of the peristaltic pumps comprises of an infusion pump configured to draw the fluid from a reservoir and create pressurized fluid for wound treatment.

31. The system according to claim 29, wherein the rotation speed of each of the peristaltic pumps is variable to control the speed and volume of aspiration.

32. The system according to claim 29, wherein the micro nozzles have a diameter in the range of 40 and 60 microns.

33. The system according to claim 29, wherein an axis of the micronozzles creates an angle of 90 degrees with respect to a surface forming the inner conical part of the applicator such that the micro-jets emanating from the nozzles of the applicator are perpendicular to the surface of the wound.

34. The system according to claim 29, wherein the application of vacuum and pressurized fluid by the negative pressure applicator to the wound surface is carried out by moving the operating handle along the wound surface by changing the speed and the direction of movement.

35. The system according to claim 29, wherein the fluid comprises one or more therapeutic fluids.

36. The system according to claim 29, wherein the applicator may be of cylindrical shape with a diameter of contact sport of 5 mm.

37. The system according to claim 29, wherein the occlusion tubes may be elastic tubes.
